(19) United States
(12) Patent Application Publication
(10) Pub. No.: US 2024/0318010 A1
(43) Pub. Date: Sep. 26, 2024

(54) ARTICLES WITH SURFACE COATING AND METHODS FOR MAKING THE SAME

(71) Applicant: MEDTRONIC, INC., Minneapolis, MN (US)

(72) Inventors: Brian Richard Upadhyaya, Woodbury, MN (US); Marc Albert Rufin, Brooklyn Park, MN (US)

(21) Appl. No.: 18/585,889

(22) Filed: Feb. 23, 2024

Related U.S. Application Data
(60) Provisional application No. 63/453,277, filed on Mar. 20, 2023.

(51) Int. Cl. C09D 5/16; A61F 2/82; C09D 133/14

(52) U.S. Cl. CPC C09D 5/1668; A61F 2/82; C09D 133/14

(57) ABSTRACT

Articles including the reaction product of an organic coating compound covalently bound to the surface thereof and methods of making the same are provided. The organic coating compound including at least one of an amine group, a hydroxyl group, or a carbonyl group. The reaction product covalently bound directly to the surface of the implantable medical device via at least one of an amine linkage, an ether linkage, or a ketone linkage.

### FIELD

The present disclosure relates to articles, such as implantable medical devices, with surfaces coated with organic coating compounds. The present disclosure further relates to methods for binding organic coating compounds to surfaces.

### SUMMARY

Embodiments described herein are directed to illustrative articles including a surface and the reaction product of an organic coating compound comprising at least one of an amine group, a hydroxyl group, or a carbonyl group. The reaction product is covalently bound directly to the surface via at least one of an amine linkage, an ether linkage, or a ketone linkage. Examples of such articles include implantable medical devices. However, other articles may also be coated using the methods of the present disclosure.

The organic coating compound may be a polymer having repeating units including the at least one of an amine group, a hydroxyl group, or a carbonyl group. The organic coating compound may be a thromboresistant polymer. The organic coating compound may further include at least one zwitterionic group. The organic coating compound may be a polymer having repeating units including at least one zwitterionic pendant group.

The amine group of the organic coating compound may be a primary amine.

The implantable medical device may be free of silane.

The medical device may be a venous stent, implantable lead, catheter, vascular implant, cardiac implant, or other blood-contacting implantable medical device.

The reaction product of the organic coating compound may be covalently bound directly to the surface of the implantable medical device without a silane primer. The reaction product of the organic coating compound may be covalently bound directly to the surface without a grafting primer.

The surface of the implantable medical device may include at least one of a metal, a ceramic, a glass, or a polymer. The surface may include at least one of nitinol, titanium, cobalt chromium, or steel.

Some embodiments described herein are directed to a method of grafting an organic coating compound to a surface. The surface may be the surface of an article, such as an implantable medical device. The method includes preparing a grafting solution including the organic coating compound. The organic coating compound includes at least one of an amine group, a hydroxyl group, or a carbonyl group. The method optionally includes activating the surface to introduce surface hydroxyl, amine, or carbonyl groups, the surface hydroxyl, amine, or carbonyl groups bound directly to the surface of the implantable medical device. The method further includes contacting the surface with the grafting solution and drying the surface. The at least one of an amine group, a hydroxyl group, or a carbonyl group becomes covalently bound to the surface hydroxyl, amine, or carbonyl groups forming at least one of an amine linkage, an ether linkage, or a ketone linkage.

The method may further include activating the surface to introduce surface hydroxyl, amine, or carbonyl groups, the surface hydroxyl, amine, or carbonyl groups bound directly to the surface.

The organic coating compound may be a polymer having repeating units including the at least one of an amine group, a hydroxyl group, or a carbonyl group. The organic coating compound may be a thromboresistant polymer. The organic coating compound may include at least one zwitterionic group.

The amine group of the organic coating compound may be a primary amine group.

The activating the surface may include hydroxylating the surface with oxygen plasma or hydroxylating the surface with an activation solution including water and a base.

The implantable medical device may be a venous stent, implantable lead, catheter, vascular implant, cardiac implant, or other blood-contacting implantable medical device.

The surface of the implantable medical device may include at least one of a metal, a ceramic, a glass, or a polymer. The surface of the implantable medical device may include at least one of nitinol, titanium, cobalt chromium, and steel.

The drying may occur in a vacuum. The drying may occur at a temperature between 50 degrees C. and 100 degrees C.

The above summary is not intended to describe each disclosed embodiment or every implementation of the present disclosure. The figures and the detailed description below more particularly exemplify illustrative embodiments.

### BRIEF DESCRIPTION OF FIGURES

The discussion below refers to the following figures, wherein the same reference number may be used to identify the similar/same subject in multiple figures. However, the use of a number to refer to a subject in a given figure is not intended to limit the component in another figure labeled with the same number. The figures are not necessarily to scale.

FIG. 1 is a schematic of an illustrative method of binding an organic coating compound to the surface of an implantable medical device.

FIG. 2A is a schematic showing a surface of an implantable medical device activated using a basic solution, exposing free hydroxyl groups.

FIG. 2B is a schematic showing the substrate surface of FIG. 2A reacted with an organic coating compound to form covalent bonds between the organic coating compound and the surface of the implantable medical device.

FIG. 3 is a schematic showing the substrate surface of FIG. 2A reacted with an illustrative organic coating compound to form covalent bonds between the illustrative organic coating compound and the surface of the substrate.

FIG. 4 is a schematic showing the surface of the implantable medical device covalently bound to the illustrative organic coating compound of FIG. 3.

FIGS. 5-7 are graphical representations showing the phosphorus content of the illustrative substrate of FIG. 3 to demonstrate grafting efficiency and stability of the illustrative method of FIG. 1.

### Definitions

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, the terms “polymer” and “polymeric material” include, but are not limited to, organic homopolymers, copolymers, such as, for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

The term “aromatic ring” is used in this disclosure to refer to a conjugated ring system of an organic compound. Aromatic rings may include carbon atoms only, or may include one or more heteroatoms, such as oxygen, nitrogen, or sulfur.

The term “alkylated” is used in this disclosure to describe compounds that are reacted to replace a hydrogen atom or a negative charge of the compound with an alkyl group, such that the alkyl group is covalently bonded to the compound.

The term “alkyl” is used in this disclosure to describe a monovalent group that is a radical of an alkane and includes straight-chain, branched, cyclic, and bicyclic alkyl groups, and combinations thereof, including both unsubstituted and substituted alkyl groups. Unless otherwise indicated, the alkyl groups typically contain from 1 to 30 carbon atoms. In some embodiments, the alkyl groups contain 1 to 20 carbon atoms, 1 to 10 carbon atoms, 1 to 6 carbon atoms, 1 to 4 carbon atoms, or 1 to 3 carbon atoms. Examples of alkyl groups include, but are not limited to, methyl, ethyl, n-propyl, n-butyl, n-pentyl, isobutyl, t-butyl, isopropyl, n-octyl, n-heptyl, ethylhexyl, cyclopentyl, cyclohexyl, cycloheptyl, etc.

The term “(meth)acrylic” refers to acrylic and methacrylic monomers, and “(meth)acrylate” refers to acrylate and methacrylate monomers.

The term “substantially” as used herein has the same meaning as “significantly,” and can be understood to modify the term that follows by at least about 90%, at least about 95%, or at least about 98%. The term “substantially free” of a particular compound means that the compositions of the present invention contain less than 1,000 parts per million (ppm) of the recited compound. In the context of the aforementioned phrases, the compositions of the present invention contain less than the aforementioned amount of the compound whether the compound itself is present in unreacted form or has been reacted with one or more other materials.

The term “not substantially” as used here has the same meaning as “not significantly,” and can be understood to have the inverse meaning of “substantially,” i.e., modifying the term that follows by not more than 25%, not more than 10%, not more than 5%, or not more than 2%.

The term “about” is used herein in conjunction with numeric values to include normal variations in measurements as expected by persons skilled in the art, and is understood to have the same meaning as “approximately” and to cover a typical margin of error, such as +5% of the stated value.

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used herein, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

Any direction referred to herein, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

### DETAILED DESCRIPTION

The present disclosure relates to surfaces coated with organic coating compounds. The surfaces may be surfaces of articles, such as implantable medical devices. The present disclosure further relates to methods for binding organic coating compounds to surfaces.

Various coatings may be applied onto material surfaces to alter the surface properties of the material. In some cases, coatings may be grafted onto the surface of the material so that the coating is chemically (e.g., covalently) bonded onto the surface. Examples of various properties that may be achieved by grafting organic coating compounds to a surface include anti-fouling, low friction, lubrication, adhesion promotion, thromboresistance, hydrophobicity, hydrophilicity, non-wetting, etc.

For example, various surfaces, such as certain medical devices (e.g., implantable devices, surgical tools) may be treated with a fouling-resistant coating to reduce biofouling (e.g., protein adsorption, thrombosis, inflammation, infection, etc.) during use. In the context of implantable medical devices, fouling-resistant coatings and reduced biofouling may be used to reduce the risk of material-induced thrombosis and infection. Implantable medical devices that may benefit from fouling-resistant coatings may include implantable medical devices, such as, for example, stents (e.g., venous stents), catheters (e.g., dialysis catheters), ventricular assist devices, prosthetic implants, vascular implants, cardiac implants, stent grafts, diabetic glucose sensors, implantable leads, and the like. In particular, blood-contacting implantable medical devices may benefit from fouling-resistant coatings. Blood-contacting implantable medical devices may be described as medical devices commonly in direct, long-term contact with blood inside a patient's body. Blood contacting implantable medical devices may include, for example, stents, catheters, ventricular assist devices, prosthetic implants, vascular implants, cardiac implants, stent grafts, diabetic glucose sensors, implantable leads, and the like. The devices may be made of various materials, including metals and polymers. In some cases, the devices include parts made of nitinol, an alloy of nickel and titanium. In some embodiments, the devices include parts made of polyurethane (e.g., thermoplastic polyurethane), such as PELLETHANE® (available from Lubrizol in Wickliffe, Ohio).

Additionally, certain medical devices or other articles may benefit from a coating in other ways. For example, coatings may beneficially alter the lubriciousness of a material, decrease friction, promote adhesion or formation of a capsule in a body, or be non-wetting, or provide a combination of two or more such properties.

Grafting surfaces (e.g., a target surface of an implantable medical device) are commonly prepared with a grafting primer coating, such as a silane coating. Silane grafting primers may include, for example, trialkoxysilanes, which have three possible binding sites for binding with the grafting surface. While silane grafting primers are commonly used in manufacturing processes to facilitate grafting of the coating compound onto the grafting surface, in such manufacturing processes approximately 30% of the processing time may be attributed to grafting of the silane primer to the grafting surface. Devices and methods are described herein which may improve manufacturing processes to covalently attach a coating compound to a surface of a substrate.

The present disclosure provides a substrate (e.g., an article or a medical device, such as an implantable medical device) with an organic coating compound grafted directly onto the substrate surface and methods of making the same without the use of a grafting primer, such as a silane primer coating. Substrates with an organic coating compound grafted directly onto the substrate surface and methods of making the same without the use of a grafting primer may be advantageous, for example, by simplifying manufacturing processes, and/or reducing manufacturing time.

According to an embodiment, the coatings of the present disclosure may be applied onto the surface of any suitable substrate, as long as the surface has or can be modified to have hydroxyl groups, amine groups, carbonyl groups, other chemical groups capable of undergoing condensation reaction, or combinations of two or more thereof. Suitable substrates include various metals, minerals, and polymers. According to an embodiment, the coating is applied onto a metal substrate, such as nitinol, titanium (e.g., sintered titanium), steel (e.g., stainless steel), nitride, cobalt, chromium, cobalt chromium, platinum, or an alloy of two or more thereof. In one or more specific embodiments, the coating is applied onto a nitinol substrate. In some embodiments, the coating is applied onto non-metal substrates, such as a polymeric substrate or a ceramic substrate. In embodiments where the coating is applied to a polymeric substrate, a polymeric substrate that is resistant to solvents may be selected. Non-limiting examples of such polymeric substrates include polycarbonate, polycarbonate-urethane, polyurethane, polyethylene, polypropylene, and the like.

The substrate may be a medical device or may be part of a medical device, such as an implantable medical device. The substrate may include a metal or a polymer. In some embodiments, the substrate is or includes nitinol.

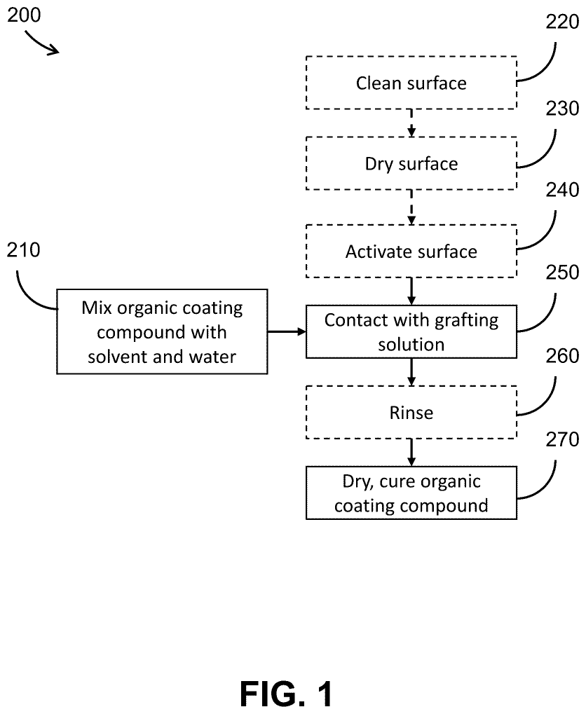

A schematic flow diagram of the method 200 for grafting an organic coating compound onto a substrate is shown in FIG. 1. A grafting solution containing the organic coating compound may be prepared, for example, by dissolving the organic coating compound in a carrier in step 210. The carrier may include a solvent or may be aqueous. The carrier may be a solution containing an organic solvent and water.

In an aqueous carrier prepared in step 210, the amount of water may be from 0.5 vol-% to 99.5 vol-% water by weight of the grafting solution. According to some embodiments, the amount of water in the grafting solution is controlled to achieve consistent grafting of the organic coating compound to the surface of the substrate. The amount of water in the grafting solution may be 0.5 vol-% or greater, 1 vol-% or greater, 2 vol-% or greater, 3 vol-% or greater, 5 vol-% or greater, 10 vol-% or greater, 20 vol-% or greater, 35 vol-% or greater, 50 vol-% or greater, or 75 vol-% or greater. The amount of water may be 98 vol-% or less, 95 vol-% or less, 85 vol-% or less, 70 vol-% or less, 50 vol-% or less, 30 vol-% or less, 15 vol-% or less, or 10 vol-% or less. The amount of water in the solution may be from 50 vol-% to 99.5 vol-% water, from 85 vol-% to 99 vol-%, or from 92 vol-% to 98 vol-% water. The amount of water in the solution may be about 95 vol-% water.

In a carrier including an organic solvent prepared in step 210, the amount of organic solvent may be from 0.5 vol-% to 80 vol-% organic solvent by weight of the grafting solution. According to some embodiments, the amount of organic solvent may be 0.5 vol-% or greater, 1 vol-% or greater, 2 vol-% or greater, 3 vol-% or greater, or 4 vol-% or greater. The amount of organic solvent may be 80 vol-% or less, 50 vol-% or less, 40 vol-% or less, 30 vol-% or less, 25 vol-% or less, 20 vol-% or less, 15 vol-% or less, or 10 vol-% or less. The amount of organic solvent in the carrier may be from 0.5 vol-% to 50 vol-% organic solvent, from 1 vol-% to 10 vol-%, or from 2 vol-% to 8 vol-% organic solvent. For example, the amount of organic solvent may be from 0.5 vol-% to 50 vol-% organic solvent, from 1 vol-% to 10 vol-%, or from 2 vol-% to 8 vol-% organic solvent by weight of the grafting solution. In one or more embodiments, the balance of the grafting solution may be organic solvent. Many organic solvents may be compatible with this approach. According to some embodiments, the organic solvent may be polar. According to some embodiments, the organic solvent is miscible or partly miscible with water. Suitable organic solvents may include isopropanol, ethanol, methanol, or the like.

The carrier used to prepare the grafting solution in step 210 may include any suitable organic solvent. The solvent may be selected based on its compatibility with the coating compound. For example, the solvent may be selected based on its ability to dissolve the coating compound. The coating compound may be an organic polymer. Suitable organic solvents used to prepare the grafting solution may include, for example, ethanol, isopropyl alcohol, methanol, acetonitrile, acetone, dimethyl sulfoxide (DMSO), dimethylformamide (DMF), tetrahydrofuran (THF), chloroform, ethyl acetate, N-methyl-2-pyrrolidone (NMP), or the like. In some embodiments, the organic solvent is ethanol. In some embodiments, the carrier includes ethanol and water. In some embodiments, the carrier is ethanol and water.

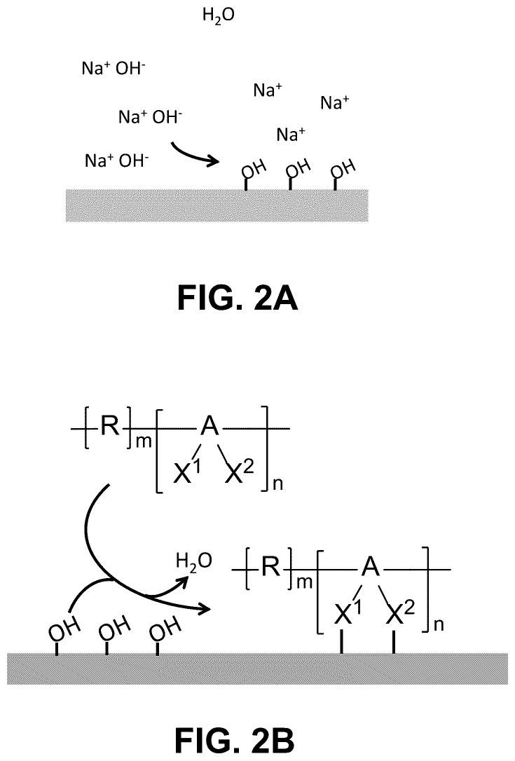

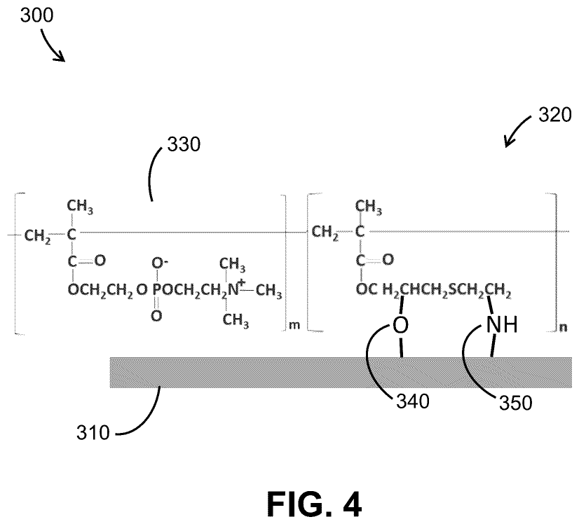

The surface of the substrate may be prepared by any suitable method for the grafting reaction. Surface preparation steps may be selected based on improving surface reactivity and avoiding or minimizing substrate degradation (e.g., corrosion, discoloration, or dissolution). Exemplary surface preparation steps 220, 230, 240 are shown in FIG. 4. An exemplary surface preparation reaction is also shown in FIG. 2A. The substrate may be prepared by cleaning the surface 220, as an example. The substrate may be prepared by drying the surface 230, as another example. As yet another example, the substrate may be prepared by a combination of cleaning the surface and drying the surface. The substrate may be cleaned to remove contamination and expose the bare substrate surface, for example, with a detergent, a solvent, UV light, heat, plasma, such as oxygen plasma, or any combination of two or more thereof. The substrate may be dried, for example, using heat or vacuum or both. According to an embodiment, the surface of the substrate may be activated 240. Activation may be used to render the surface capable of undergoing condensation reactions with the organic coating compound. Activation of the surface may include forming hydroxyl groups on (i.e., hydroxylating) the surface (see FIG. 2A), as an example. As another example, activation of the surface may additionally or alternatively include forming amine groups (e.g., primary amine groups) on the surface. As yet another example, activation of the surface may include forming carbonyl groups on the surface. The substrate may include a metal, such as nitinol, titanium, cobalt chromium, steel, or another suitable substrate. The surface may be activated using any suitable method. The surface may be activated by hydroxylation using oxygen plasma, as an example. As another example, the surface may be activated by hydroxylation with a base, such as sodium hydroxide, potassium hydroxide, or another base in an aqueous solution. The base may be sodium hydroxide. In one or more embodiments, the surface is nitinol and is activated by hydroxylation with sodium hydroxide as the base. If the substrate is a material that is already or inherently capable of undergoing condensation reactions with the organic coating compound, the activation step may be omitted. For example, if the substrate is a material that already or inherently includes surface hydroxyl groups, amine groups, or carbonyl groups, the activation step may be omitted.

The surface of the substrate (e.g., the activated surface, such as an activated nitinol surface) is then contacted with the prepared grafting solution in step 250. The contacting may be performed in any suitable way. For example, contacting the surface of the substrate with the grafting solution may include immersion, thin film deposition, dip coating, spraying, brushing, or combinations of two or more thereof. According to an embodiment, the surface of the substrate may be contacted with the grafting solution for a predetermined amount of time. The amount of contact time may be selected based on concentration of the organic coating compound in the grafting solution, as an example. In some examples, the amount of contact time may be selected based on reactivity of the surface of the substrate. The surface of the substrate may be immersed in the grafting solution for between 0.5 seconds and 10 minutes. For example, the surface of the substrate may be immersed in the grafting solution for 0.5 second or more, 1 second or more, 10 seconds or more, 30 seconds or more, 1 minute or more, 2 minutes or more, 3 minutes or more, 4 minutes or more, 5 minutes or more, 10 minutes or more, or 15 minutes or more. The surface of the substrate may be immersed in the grafting solution for 10 minutes or less, 9 minutes or less, 8 minutes or less, 7 minutes or less, 6 minutes or less, 5 minutes or less, 2 minutes or less, 1 minute or less, 30 seconds or less, 10 seconds or less, or 5 seconds or less. The surface of the substrate may be contacted with the grafting solution for a time period of 0.5 seconds to 10 minutes, 10 seconds to 5 minutes, or 30 seconds to 2 minutes. The surface of the substrate may be contacted with the grafting solution for a time period of about 1 minute.

The organic coating compound may be grafted onto the surface in a condensation reaction with free hydroxyl groups present on a surface of the substrate. An exemplary grafting reaction is shown in FIG. 2B.

Methods suitable to improving the efficiency of condensation reactions may be used to improve the efficiency of organic coating compound grafting. According to an embodiment, the reaction may occur at an elevated temperature. Increasing the reaction temperature may improve the efficiency of organic coating compound grafting. The reaction temperature may be 20° C. or greater, 35° C. or greater, 50° C. or greater, 75° C. or greater, 90° C. or greater, 100° C. or greater or 130° C. or greater. The reaction temperature may be 150° C. or less, 125° C. or less, 100° C. or less, 60 C or less, or 40° C. or less. The reaction temperature may be between 90° C. and 150° C. The reaction temperature may be the boiling point of water, which may be affected by conditions such as the ambient pressure. The reaction temperature may be the boiling point of the grafting solution, which may be affected by conditions such as the composition of the grafting solution or ambient pressure.

According to an embodiment, the reaction may occur at reduced pressure. The reaction may occur in a vacuum, for example. Performing the reaction at reduced pressure may improve the efficiency of organic coating compound grafting to the substrate surface. The reaction may occur in a pressure of 0 atm (atmospheres) or greater, 0.1 atm or greater, 0.2 atm or greater, 0.3 atm or greater, 0.5 atm or greater, 0.6 atm or greater, or 0.8 atm or greater. The reaction may occur in a pressure of 1 atm or less, 0.9 atm or less, 0.8 atm or less, 0.6 atm or less, 0.5 atm or less, 0.3 atm or less, or 0.1 atm or less. The reaction may occur at between 0 atm and 1 atm or 0.1 atm to 0.9 atm.

According to an embodiment, a thin film deposition technique is used to graft the organic coating compound onto the surface. Thin film deposition techniques typically result in a thin, uniform layer of a compound on a substrate. This may advantageously improve the uniformity of the coating formed.

The organic coating compound may be a polymeric or oligomeric compound. The organic coating compound may include two or more types of functional moieties. The first functional moiety may be a grafting moiety capable of undergoing condensation reactions with the surface of the substrate. The second functional moiety may be any suitable functional moiety capable of providing a desired property. The organic coating compound may include further functional moieties, either grafting moieties, moieties providing desired functionality, or both.

In the exemplary grafting reaction shown in FIG. 2B, the organic coating compound is represented by formula I:

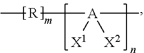

where R is a functional group that provides the organic coating compound with the desired property,

A is a grafting moiety,

X¹ and X² are independently reactive groups capable of covalently bonding with reactive groups on the surface, and

n and m are integers independently selected from 1 to 1,000.

Exemplary reactive groups X¹ and X² include hydroxyl groups and amines. Exemplary functional moieties R are further discussed below. The functional moiety R and grafting moiety A may each be repeating units of a polymeric coating compound. According to an embodiment, a plurality of grafting moieties A of the polymeric coating compound bond with the surface through one or both of the reactive groups X¹ and X².

The grafting moiety may include one or more reactive groups capable of undergoing condensation reactions with the hydroxyl, amine, or carbonyl surface groups of the substrate (e.g., after the surface of the substrate has been activated or the surface of a substrate that is already or inherently capable of undergoing condensation reactions with the organic coating compound). While formula I and FIG. 2B each shows a grafting moiety A including two reactive groups X¹ and X², the grafting moiety may include more or fewer reactive groups, such as one reactive group or three or more reactive groups per grafting moiety. The coating compound may also include multiple different grafting moieties, where each grafting moiety independently includes one or more reactive groups X¹, X², X³, etc. The reactive groups X¹, X², X³, etc. on the multiple grafting moieties may be the same or different. The grafting moiety may include any reactive groups capable of undergoing a condensation reaction with the surface of the substrate to form a covalent bond between the organic coating compound and the surface of the substrate. Suitable grafting moiety reactive groups may include, for example, amine groups (e.g., primary, secondary, or tertiary amine groups), hydroxyl groups, and carbonyl groups. The organic coating compound may be covalently bound to the surface through one or more, or two or more covalent bonds. In some cases, the organic coating compound is covalently bound to the surface through at least two covalent bonds that are different from each other. Such covalent bonds may include an ether bond, an amide bond, a ketone bond, or a combination of two or more thereof. In one or more specific embodiments, one or more of the grafting moiety reactive groups may be pendant groups of the organic coating compound. In some embodiments, the organic coating compound may be a polymer including the grafting moiety. Such a polymer may have repeating units including the grafting moiety. In one or more specific embodiments, the organic coating compound may be a polymer with repeating units including at least one of an amine functional group, a hydroxyl group, or a carbonyl group. In some cases, the organic coating compound is covalently bound to the surface through at least two covalent bonds per repeating unit.

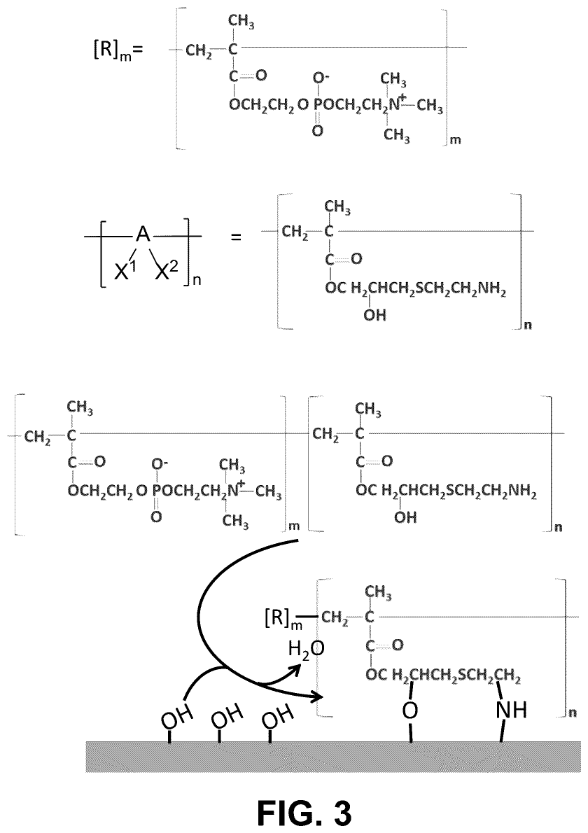

The second functional moiety (e.g., the functional moiety R) may include one or more functional groups that can provide a desired property to the substrate. That is to say, the second functional moiety may be selected to achieve a desirable surface property. Desired properties may include, for example, one or more of anti-fouling, thromboresistance, low friction, lubrication, adhesion promotion, hydrophobicity, hydrophilicity, non-wetting, and the like. In some embodiments, the organic coating compound may be a polymer including the second functional moiety. Such a polymer may have repeating units including the second functional moiety. According to some embodiments, the organic coating compound may be a thromboresistant polymer, such as, for example, LIPIDURE (available from NOF AMERICA). A schematic reaction diagram of a surface coating process including LIPIDURE as the coating compound is shown in FIG. 3.

According to an embodiment, the second functional moiety may be a zwitterionic moiety. In one or more specific embodiments, the organic coating compound may be a polymer with repeating units including at least one zwitterionic pendant group. In some embodiments, the second functional moiety may include, for example, a phospholipid moiety, and may further include phosphorylcholine. In some specific embodiments, the second functional moiety may confer thromboresistance to blood-contacting surfaces of an article (e.g., of an implantable medical device). Examples of suitable phosphorylcholines include 2-methacryloyloxyethyl phosphorylcholine, 2-acryloyloxyethyl phosphorylcholine, and phosphorylcholines based on monomers such as 2-(meth)acryloyloxyethyl-2′-(trimethylammonio)ethyl phosphate, 3-(meth)acryloyloxypropyl-2′-(trimethylammonio)ethyl phosphate, 4-(meth)acryloyloxybutyl-2′-(trimethylammonio)ethyl phosphate, 5-(meth)acryloyloxypentyl-2′-(trimethylammonio)ethyl phosphate, 6-(meth)acryloyloxyhexyl-2′-(trimethylammonio)ethyl phosphate, 2-(meth)acryloyloxyethyl-2′-(tricthylammonio)ethyl phosphate, 2-(meth)acryloyloxyethyl-2′-(tripropylammonio)ethyl phosphate, 2-(meth)acryloyloxyethyl-2′-(tributylammonio)ethyl phosphate, 2-(meth)acryloyloxypropyl-2′-(trimethylammonio)ethyl phosphate, 2-(mcth)acryloyloxybutyl-2′-(trimethylammonio)ethyl phosphate, 2-(meth)acryloyloxypentyl-2′-(trimethylammonio)ethyl phosphate, 2-(meth)acryloyloxyhexyl-2′-(trimethylammonio)ethyl phosphate, 2-(meth)acryloyloxyethyl-3′-(trimethylammonio)propyl phosphate, 3-(meth)acryloyloxypropyl-3′-(trimethylammonio)propyl phosphate, 4-(meth)acryloyloxybutyl-3′-(trimethylammonio)propyl phosphate, 5-(meth)acryloyloxypentyl-3′-(trimethylammonio)propyl phosphate, 6-(meth)acryloyloxyhexyl-3′-(trimethylammonio)propyl phosphate, 2-(meth)acryloyloxyethyl-4-(trimethylammonio)butyl phosphate, 3-(meth)acryloyloxypropyl-4′-(trimethylammonio)butyl phosphate, 4-(meth)acryloyloxybutyl-4′-(trimethylammonio)butyl phosphate, 5-(meth)acryloyloxypentyl-4′-(trimethylammonio)butyl phosphate, 6-(meth)acryloyloxyhexyl-4′-(trimethylammonio)butyl phosphate, and combinations thereof.

In some embodiments, the second functional moiety confers an anti-fouling property to the surface of the substrate. Organic coating compounds including a second functional moiety conferring the anti-fouling property may be grafted, for example, to implantable medical devices or to blood contacting surfaces of medical devices. Examples of organic coating compounds with a suitable anti-fouling properties include polyethylene glycol (PEG), polyethylene oxide (PEO), quaternary ammonium, and heparin.

The organic coating compound may have any suitable ratio of first functional moieties (e.g., the grafting moiety A) to second functional moieties (e.g., the functional moiety R). Suitable ratios may be selected based on factors such as the relative molecular weights of each moiety, the molecular weight of the organic coating compound, the desired magnitude of effect from the second functional moieties, the desired grafting efficiency of the organic coating compound, or the grafting efficiency of the first functional moieties on the substrate material, as a few examples. Suitable ratios may include, for example, between 1:20 and 20:1 or between 1:10 and 10:1. In one embodiment, the ratio may be approximately 1:6. As further examples, suitable ratios may include 1:20 or greater, 1:15 or greater, 1:10 or greater, 1:6 or greater, 1:2 or greater, 1:1 or greater, 2:1 or greater, 6:1 or greater, 10:1 or greater, 15:1 or greater, or 20:1 or greater, and/or 20:1 or less, 15:1 or less, 10:1 or less, 6:1 or less, 2:1 or less, 1:1 or less, 1:2 or less, 1:6 or less, 1:10 or less, 1:15 or less, or 1:20 or less.

Referring again to FIG. 1, after contacting with the grafting solution, the surface of the substrate is optionally rinsed 260. The surface may be rinsed using the same solvent or combination of solvents used to dissolve the organic coating compound (e.g., in step 210), or it may be rinsed in a different solvent. Suitable solvents for rinsing the surface may include water, as an example. Examples of suitable solvents for rinsing the surface may include solvents in which the organic coating compound is soluble. In some embodiments, the rinsing step may be omitted.

Following contacting with the grafting solution and optionally rinsing, the surface may be dried, and the grafting bonds may be cured 270. During curing 270, the organic coating compound may undergo a condensation reaction with the surface of the substrate, and thereby become covalently bound directly to the grafting surface. According to an embodiment, curing 270 includes drying the surface under vacuum. According to an embodiment, curing 270 includes drying the surface at a high temperature. In one or more specific embodiments, curing 270 may include drying the surface at a high temperature and under vacuum. The drying temperature may be 50° C. or more, 75° C. or more, 90° C. or more, 300° C. or more, 325° C. or more, or 350° C. or more. The drying temperature may be 300° C. or less, 375° C. or less, 350° C. or less, 325° C. or less, 300° C. or less, 80° C. or less, or 70° C. or less. The drying temperature may be, for example, from 90° C. to 150° C., such as approximately 100° C. Lower temperatures, for example as low as ambient temperature (about 20° C. to 25° C.), may be used if drying is performed under vacuum.

According to an embodiment, the methods of the present disclosure may be used to prepare coated substrates. The coated substrate may include an organic coating compound grafted onto the surface of the substrate. The substrate may include a surface.

The grafting density of the organic coating compound on the surface correlates with elemental analysis of the surface. In some embodiments, the thickness of the organic coating compound on the surface may correlate with the element analysis of the surface, such as where the coating thickness is less than or equal to the depth of penetration of the surface analysis. Elemental analysis (e.g., concentration of an element present in the organic coating compound) of the surface may be performed, for example, by using XPS/ESCA (X-ray Photoelectron Spectroscopy (XPS) also known as Electron Spectroscopy for Chemical Analysis (ESCA)) or ellipsometry. The concentration, which correlates with grafting density, may be expressed as an atomic percentage of the target element on the surface. Suitable analytical techniques for characterizing grafting efficiency or stability may include Time-of-Flight Secondary Ion Mass Spectrometry (TOF-SIMS), as another example.

The target element for elemental analysis may be selected based on the presence of the target element in the organic coating compound, as an example. As another example, the target element may be selected based on the absence or low occurrence of the target element in the substrate and other possible sources of interference. In one or more examples, the target element may be phosphorus, such as for elemental analysis of a substrate that does not include phosphorus (e.g., nitinol), coated with an organic coating compound that does include phosphorus (e.g., a phosphorylcholine such as LIPIDURE). As another example, the target element may be nitrogen, such as for elemental analysis of a substrate that does not include nitrogen (e.g., nitinol) coated with an organic coating compound that does include nitrogen (such as a phosphorylcholine such as LIPIDURE).

The coating may exhibit an elemental concentration, measured as the elemental concentration of a target element present in the organic coating compound, of 0.1 atomic % or more, 0.5 atomic % or more, 1 atomic % or more, 1.5 atomic % or more, 2 atomic % or more, or 5 atomic % or more. There may not be a desired upper limit for the elemental concentration of the coating compound. The coating may exhibit an elemental concentration of a maximum value, indicating a thick, dense coating. The maximum value may be described as a function of the percentage of the number of atoms for the target element versus the total number of atoms in the organic coating compound. In practice, the elemental concentration of, for example, phosphorus in a phosphorylcholine coating, may be 8 atomic % or less, 6 atomic % or less, 4 atomic % or less, 2 atomic % or less, or 1 atomic % or less.

According to an embodiment, the amount of coating is substantially similar to a coating prepared with the same coating compound and a silane treatment of the surface. For example, the amount of coating prepared according to methods of the present disclosure may be 70% or more, 75% or more, 80% or more, or 85% or more of a coating prepared with the same coating compound and a silane treatment of the surface.

An illustrative implantable medical device 300 prepared according to methods for grafting an organic coating compound onto a substrate is shown in FIG. 1. The implantable medical device 300 includes a surface 310. The surface 310 may be made of various materials, including metals, polymers, and combinations of two or more thereof. The surface may be made of metal, such as nitinol, titanium (e.g., sintered titanium), steel (e.g., stainless steel), nitride, cobalt, chromium, cobalt chromium, platinum, or an alloy or combination of two or more thereof. In some embodiments, the devices include parts made of nitinol, an alloy of nickel and titanium.

The illustrative implantable medical device 300 further includes the reaction product 320 of an organic coating compound, as described in detail above. The organic coating compound may include a grafting moiety. The grafting moiety may include any suitable functional groups. Suitable functional groups may include, for example, at least one amine group, at least one hydroxyl group, at least one carbonyl group, or a combination of two or more thereof.

The reaction product 320 may include a second functional moiety 330, as described in detail above. The second functional moiety may be any suitable functional moiety capable providing a desired property to the illustrative implantable medical device 300.

The reaction product 320 of the organic coating compound is covalently bound directly to the surface 310 of the illustrative implantable medical device 300. The reaction product 320 may be covalently bound by at least one of an amine linkage 350, at least one of an ether linkage 340, or a combination of two or more thereof.

### Aspects

A list of illustrative aspects is provided below.

Aspect 1 is an implantable medical device comprising:
a surface; and
the reaction product of an organic coating compound comprising at least one of an amine group, a hydroxyl group, or a carbonyl group, the reaction product covalently bound directly to the surface via at least one of an amine linkage, an ether linkage, or a ketone linkage.

Aspect 2 is the implantable medical device of any one of aspects 1 and 3-12, wherein the organic coating compound is a polymer comprising repeating units comprising the at least one of an amine group, a hydroxyl group, or a carbonyl group.

Aspect 3 is the implantable medical device of any one of aspects 1-2 and 4-12, wherein the organic coating compound is a thromboresistant polymer.

Aspect 4 is the implantable medical device of any one of aspects 1-3 and 5-12, wherein the organic coating compound further comprises at least one zwitterionic group.

Aspect 5 is the implantable medical device of any one of aspects 1-4 and 6-12, wherein the organic coating compound is a polymer and further comprises repeating units comprising at least one zwitterionic pendant group.

Aspect 6 is the implantable medical device of any one of aspects 1-5 and 7-12, wherein the amine group of the organic coating compound is a primary amine.

Aspect 7 is the implantable medical device of any one of aspects 1-6 and 8-12, wherein the implantable medical device is free of silane.

Aspect 8 is the implantable medical device of any one of aspects 1-7 and 9-12, wherein the device is a venous stent, implantable lead, catheter, vascular implant, cardiac implant, or other blood-contacting implantable medical device.

Aspect 9 is the implantable medical device of any one of aspects 1-8 and 10-12, wherein the reaction product of the organic coating compound is covalently bound directly to the surface without a silane primer.

Aspect 10 is the implantable medical device of any one of aspects 1-9 and 11-12, wherein the reaction product of the organic coating compound is covalently bound directly to the surface without a grafting primer.

Aspect 11 is the implantable medical device of any one of aspects 1-10 and 12, wherein the surface comprises at least one of a metal, a ceramic, a glass, or a polymer.

Aspect 12 is the implantable medical device of any one of aspects 1-11, wherein the surface comprises at least one of nitinol, titanium, cobalt chromium, or steel.

Aspect 13 is a method of grafting an organic coating compound to a surface of an implantable medical device, the method comprising:

Preparing a grafting solution comprising the organic coating compound, the organic coating compound comprising at least one of an amine group, a hydroxyl group, or a carbonyl group; optionally activating the surface to introduce surface hydroxyl, amine, or carbonyl groups, the surface hydroxyl, amine, or carbonyl groups bound directly to the surface;
contacting the surface with the grafting solution; and
drying the surface;
wherein the at least one of an amine group, a hydroxyl group, or a carbonyl group becomes covalently bound to the surface forming at least one of an amine linkage, an ether linkage, or a ketone linkage.

Aspect 14 is the method of any one of aspects 13 and 15-24, further comprising activating the surface to introduce surface hydroxyl, amine, or carbonyl groups, the surface hydroxyl, amine, or carbonyl groups bound directly to the surface.

Aspect 15 is the method of any one of aspects 13-14 and 16-24, wherein the organic coating compound is a polymer comprising repeating units comprising the at least one of an amine group, a hydroxyl group, or a carbonyl group.

Aspect 16 is the method of any one of aspects 13-15 and 17-24, wherein the organic coating compound is a thromboresistant polymer.

Aspect 17 is the method of any one of aspects 13-16 and 18-24, wherein the organic coating compound comprises at least one zwitterionic group.

Aspect 18 is the method of any one of aspects 13-17 and 19-24, wherein the amine group of the organic coating compound is a primary amine group.

Aspect 19 is the method of any one of aspects 13-18 and 20-24, wherein the activating the surface comprises hydroxylating the surface with oxygen plasma or hydroxylating the surface with an activation solution comprising water and a base.

Aspect 20 is the method of any one of aspects 13-19 and 21-24, wherein the implantable medical device is a venous stent, implantable lead, catheter, vascular implant, cardiac implant, or other blood-contacting implantable medical device.

Aspect 21 is the method of any one of aspects 13-20 and 22-24, wherein the surface comprises at least one of a metal, a ceramic, a glass or a polymer.

Aspect 22 is the method of any one of aspects 13-21 and 23-24, wherein the surface comprises at least one of nitinol, titanium, cobalt chromium, and steel.

Aspect 23 is the method of any one of aspects 13-22 and 24, wherein the drying occurs in a vacuum.

Aspect 24 is the method of any one of aspects 13-23, wherein the drying occurs at a temperature between 50 degrees C. and 100 degrees C.

### EXAMPLES

TABLE 1
Reagents used in the Examples

| Reagent | Source |
|---|---|
| Sodium hydroxide (NaOH) | Fisher Scientific |
| Deionized (DI) water | house made |
| Nitinol coupons | Fort Wayne Metals in Fort Wayne, IN |
| (3-GLYCIDOXYPROPYL)TRIETHOXYSILANE | Gelest, Inc. in Morrisville, PA |
| 100% ethanol | Sigma Aldrich, Fisher Scientific |
| LIPIDURE ®-NH01 | NOF Corporation in Tokyo, Japan |
| TRIS buffer | Thermo Fisher Scientific |
| PELLETHANE ® TPU 55D | Lubrizol |

The grafting efficiency of the method of the present disclosure was tested for LIPIDURE®-NH01 grafted into a nitinol surface. Similarly, the grafting efficiency of the method of the present disclosure was tested for LIPIDURE®-NH01 grafted onto a PELLETHANE surface. Samples were prepared to compare the grafting efficiency of LIPIDURE®-NH01 by the method of the present disclosure to the grafting efficiency of LIPIDURE®-NH01 by a method using a grafting primer coating. The first method is consistent with the methods described herein. In the second method, as a comparative method, a silane primer coating was grafted onto the activated substrate surface before contacting the substrate surface with the organic coating compound grafting solution.

Additionally, samples were prepared to compare the grafting efficiency of LIPIDURE®-NH01 by the method of the present disclosure to the grafting efficiency of poly(phosphorylcholine-methacrylate) (poly(PC-methacrylate)) by the method of the present disclosure. In the third, as another comparative method, the substrate was prepared consistent with the methods described herein before contacting the substrate surface with the poly(PC-methacrylate). Poly(PC-methacrylate) lacks the grafting moiety of the organic coating compound described herein.

| Grafting Treatment | Substrate | Sample ID |
|---|---|---|
| Organic coating compound | Nitinol | Nitinol 1 |
| Silane primer and organic coating | Nitinol | Nitinol C1 |
| compound (comparative) | | |
| Poly(PC-methacrylate) (comparative) | Nitinol | Nitinol C2 |
| Clean, untreated (control) | Nitinol | Nitinol Control |
| Organic coating compound | PELLETHANE | Pellethane 1 |
| Silane primer and organic coating | PELLETHANE | Pellethane C1 |
| compound (comparative) | | |

### Comparative Silane Primer Grafting Solution

The silane primer grafting solution was prepared with 100% ethanol, hydrochloric acid (HCl), and alkoxysilane. The silane primer grafting solution was neutralized by addition of sodium hydroxide (NaOH).

### Substrate Preparation

Nitinol coupons were activated by immersing the coupons in 20% aqueous NaOH solution for 90 minutes and rinsing in 50% ethanol for 2 minutes.

PELLETHANE coupons were activated by treatment with oxygen plasma for 2 minutes.

### Comparative Silanization Treatment

After substrate preparation, the comparative silanization treatment sample coupons (Nitinol C1 coupons and Pellethane C1 coupons) were immersed in the prepared silane primer grafting solution. The prepared silane primer grafting solution with the coupons was mixed on an orbital shaker for 5 minutes. The coupons were withdrawn slowly from the silane primer grafting solution. The coupons were then cured in an oven at 110° C. for 1 hour. After curing, the coupons were cooled to room temperature. The coupons were placed in ethanol and sonicated for 3 minutes, then transferred into DI water and sonicated for 3 minutes. The coupons were dried with compressed nitrogen.

### Organic Coating Compound Treatment

After substrate preparation (Nitinol 1 coupons and Pellethane 1 coupons) and after silanization treatment (Comparative Nitinol C1 coupons and Pellethane C1 coupons), the coupons were immersed in a solution of LIPIDURE®-NH01. After immersion, the coupons were cured at 80° C. for 30 minutes, then cooled to room temperature. After cooling, the coupons were rinsed in deionized (DI) water for 10 minutes. After rinsing, the coupons were dried with compressed nitrogen.

### Comparative Coating Compound Treatment

After substrate preparation, the comparative coating compound treatment coupons (Nitinol C2 coupons) were immersed in a solution of poly(2-methacryloyloxyethyl phosphorylcholine) in DI water. After immersion, the coupons were cured at 80° C. for 30 minutes, then cooled to room temperature. After cooling, the coupons were rinsed with DI water for 10 minutes. After rinsing, the coupons were dried with compressed nitrogen.

### Immersion in Biochemical Buffer

After curing, the coupons were immersed in TRIS buffer and soaked for 2 weeks or 12 weeks at 25° C. to stress the durability of the grafted organic coating compound and verify that it was covalently bonded based on comparison with the silane primer-treated comparative samples Nitinol C1 and Pellethane C1.

### Elemental Analysis

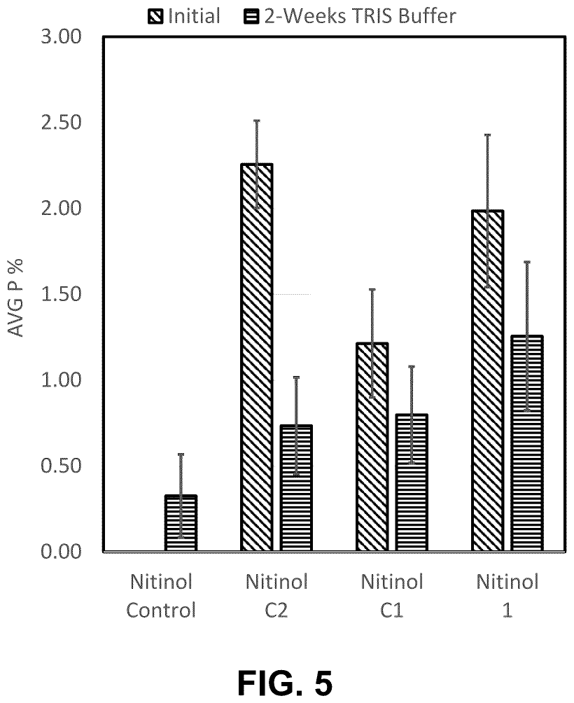

Grafting efficiency was evaluated by measuring elemental composition at the surface of the coupons by XPS. Phosphorus is unique to LIPIDURE®-NH01 and poly(PC-methacrylate), and is not found in the compositions of other materials used, such as the coupons, the TRIS buffer, and the silane primer grafting solution. Therefore, the quantity of Phosphorus detected at the surface of the Nitinol 1, Pellethane 1, Nitinol C1, and Pellethane C1 coupons is correlated with the quantity of LIPIDURE®-NH01 at the surfaces of the Nitinol 1, Pellethane 1, Nitinol C1, and Pellethane C1 coupons. Similarly, the quantity of Phosphorus detected at the surface of the Nitinol C2 coupons is correlated with the quantity of Poly(PC-methacrylate) at the surface of the Nitinol C2 coupons. Uncoated nitinol coupons and uncoated PELLETHANE coupons were used as controls. The results are shown in FIGS. 5-7.

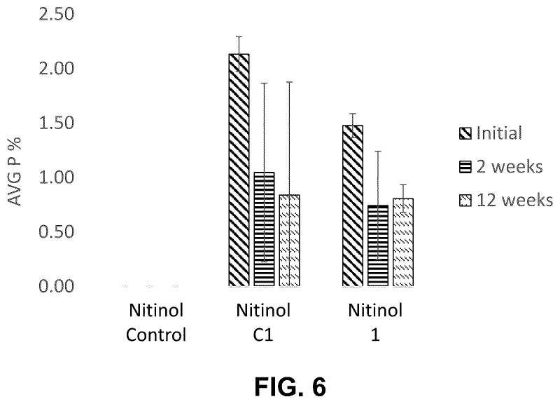

A graphical representation of element analysis results of uncoated nitinol coupons (Nitinol Control), nitinol coupons treated with poly(PC-methacrylate) (Nitinol C2), nitinol coupons treated with LIPIDURE®-NH01 with the silane primer (Nitinol C1), and nitinol coupons treated with LIPIDURE®-NH01 without the silane primer (Nitinol 1) is shown in FIG. 5. The bar graph shows average atomic percent of Phosphorus detected at the surface of the coupons after initial treatment and after immersion in the TRIS buffer for two weeks. Similar to the graphical representation of FIG. 5, a graphical representation of elemental analysis results of Nitinol Control coupons, Nitinol C1 coupons, and Nitinol 1 coupons is shown in FIG. 6. The bar graph shows average atomic percent of Phosphorus detected at the surface of the coupons after initial treatment, after immersion in the TRIS buffer for two weeks, and after immersion in the TRIS buffer for twelve weeks.

It was observed both initially and after two weeks of immersion in the TRIS buffer, that similar atomic percent of Phosphorus was detected at the surface of the coupons for the Nitinol 1 and Nitinol C1 test groups. It was further observed for both the Nitinol 1 and Nitinol C1 test groups, that similar atomic percent of Phosphorus was detected after two weeks and after twelve weeks of immersion in the TRIS buffer. The nitinol C2 test group was observed to have a significant drop in atomic percent of Phosphorus from the initial measurement after two weeks of immersion in the TRIS buffer. It was concluded that treatment with LIPIDURE®-NH01 without the silane primer, according to the embodiments of the present disclosure, provided effective grafting to the substrate with long-term stability. It was further concluded that treatment with LIPIDURE®-NH01, both with and without the silane primer, exhibit the same grafting reaction (i.e., condensation reaction). It was still further concluded that treatment with poly(PC-methacrylate) did not provide effective grafting to the substrate due to the absence of functional groups capable of condensation reactions with the substrate.

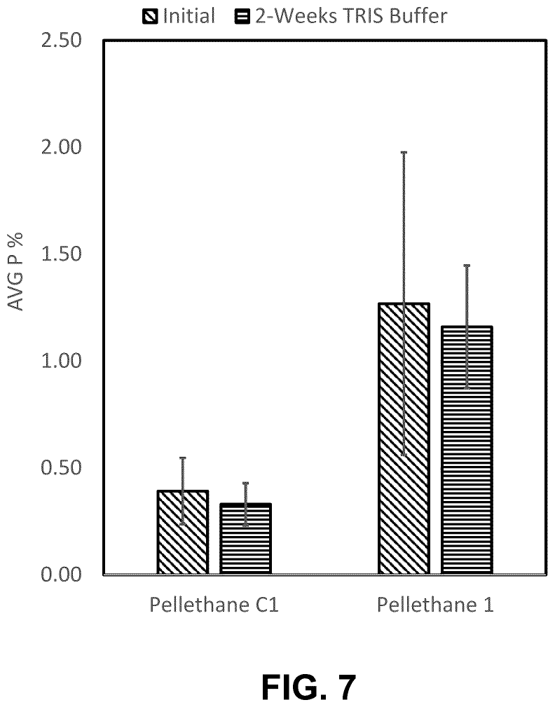

A graphical representation of elemental analysis results of PELLETHANE coupons treated with LIPIDURE®-NH01 with the silane primer (Pellethane C1) and PELLETHANE coupons treated with LIPIDURE®-NH01 without the silane primer (Pellethane 1) is shown in FIG. 7. The bar graph shows average atomic percent of Phosphorus detected at the surface of the coupons after initial treatment and after immersion in the TRIS buffer for two weeks.

It was observed for the Pethane C1 and the Pellethane 1 test groups that the atomic percent of Phosphorus detected at the surface of the coupons was unchanged from initial levels after two weeks of immersion in TRIS buffer. It was concluded that treatment with LIPIDURE®. NH01 without the silane primer, according to embodiments of the present disclosure, provided effective grafting to the PELLETHANE substrate.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. An implantable medical device comprising:
a surface; and
a reaction product of an organic coating compound comprising at least one of an amine group, a hydroxyl group, or a carbonyl group, the reaction product covalently bound directly to the surface via at least one of an amine linkage, an ether linkage, or a ketone linkage.

2. The implantable medical device of claim 1, wherein the organic coating compound is a polymer comprising repeating units comprising the at least one of an amine group, a hydroxyl group, or a carbonyl group.

3. The implantable medical device of claim 1, wherein the organic coating compound is a thromboresistant polymer.

4. The implantable medical device of claim 1, wherein the organic coating compound further comprises at least one zwitterionic group.

5. The implantable medical device of claim 1, wherein the organic coating compound is a polymer and further comprises repeating units comprising at least one zwitterionic pendant group.

6. The implantable medical device of claim 1, wherein the amine group of the organic coating compound is a primary amine.

7. The implantable medical device of claim 1, wherein the implantable medical device is free of silane.

8. The implantable medical device of claim 1, wherein the device is a venous stent, implantable lead, catheter, vascular implant, cardiac implant, or other blood-contacting implantable medical device.

9. The implantable medical device of claim 1, wherein the reaction product of the organic coating compound is covalently bound directly to the surface without a silane primer.

10. The implantable medical device of claim 1, wherein the reaction product of the organic coating compound is covalently bound directly to the surface without a grafting primer.

11. The implantable medical device of claim 1, wherein the surface comprises at least one of a metal, a ceramic, a glass, a polymer, nitinol, titanium, cobalt chromium, or steel.

12. A method of grafting an organic coating compound to a surface of an implantable medical device, the method comprising:
preparing a grafting solution comprising the organic coating compound, the organic coating compound comprising at least one of an amine group, a hydroxyl group, or a carbonyl group;
contacting the surface with the grafting solution; and
drying the surface;
wherein the at least one of an amine group, a hydroxyl group, or a carbonyl group becomes covalently bound to the surface forming at least one of an amine linkage, an ether linkage, or a ketone linkage.

13. The method of claim 12, further comprising activating the surface to introduce surface hydroxyl, amine, or carbonyl groups, the surface hydroxyl, amine, or carbonyl groups bound directly to the surface.

14. The method of claim 13, wherein the activating the surface comprises hydroxylating the surface with oxygen plasma or hydroxylating the surface with an activation solution comprising water and a base.

15. The method of claim 12, wherein the organic coating compound is a polymer comprising repeating units comprising the at least one of an amine group, a hydroxyl group, or a carbonyl group.

16. The method of claim 12, wherein the organic coating compound is a thromboresistant polymer.

17. The method of claim 12, wherein the organic coating compound comprises at least one zwitterionic group.

18. The method of claim 12, wherein the implantable medical device is a venous stent, implantable lead, catheter, vascular implant, cardiac implant, or other blood-contacting implantable medical device.

19. The method of claim 12, wherein the surface comprises at least one of a metal, a ceramic, a glass, a polymer, nitinol, titanium, cobalt chromium, or steel.

20. The method of claim 12, wherein the drying occurs in a vacuum or at a temperature between 50 degrees C. and 150 degrees C., or both.
